(19) United States
(12) Patent Application Publication
(10) Pub. No.: US 2020/0138885 A1
(43) Pub. Date: May 7, 2020

(54) LIPOPHILIC ACTIVE ORAL FILM FORMULATION AND METHOD OF MAKING THE SAME

(71) Applicant: Intelgenx Corp., St-Laurent (CA)

(72) Inventors: Nadine Paiement, St-Laurent (CA); Justin W. Conway, Carignan (CA); Carolin Madwar, Montreal (CA)

(73) Assignee: Intelgenx Corp., Saint-Laurent (CA)

(21) Appl. No.: 16/675,867

(22) Filed: Nov. 6, 2019

Related U.S. Application Data
(60) Provisional application No. 62/756,341, filed on Nov. 6, 2018.

(51) Int. Cl. A61K 36/185; A61K 9/70; A61K 9/00

(52) U.S. Cl. CPC A61K 36/185; A61K 9/0056; A61K 9/7007

(57) ABSTRACT

Disclosed is a description and methods for formulating oral films containing lipophilic active ingredient(s), more particularly lipophilic active having a positive logP. The method involves dispersing the lipophilic active(s) in a carrier oil and uniformly distributing them as emulsified oil droplets into a polymer matrix. The methods reported here produce oral films containing a stable emulsion with up to 40% oil phase. The oil phase consists of the carrier oil and lipophilic active(s). This offers the possibility to enhance the amount of lipophilic actives to be included in the film formulation while preserving the film characteristics. The resulting oral films offer a standardized dosage form for lipophilic actives as well as easier and more convenient administration, transportation, handling, and storage.

### CROSS-REFERENCE TO RELATED APPLICATIONS

This application claims priority to U.S. Provisional Application No. 62/756,341, filed Nov. 6, 2018, and which is hereby incorporated by reference in its entirety.

### FIELD OF THE DISCLOSURE

This disclosure concerns oral films formulation for oral films (OFs) containing lipophilic active ingredients, and methods of making oral films containing lipophilic active ingredients.

### BACKGROUND OF THE DISCLOSURE

The cannabis plant has a long history of medicinal use, with promising clinical applications in managing symptoms associated with cancer, acquired immune deficiency syndrome (AIDS), anxiety, depression, post-traumatic stress disorder, and more. A9-Tetrahydrocannabinol (THC) is a primary active ingredient of Cannabis and is responsible for many pharmacological effects of the plant. To date, the THC clinical applications approved by the Food and Drug Administration (FDA) are for the control of anorexia associated with weight loss in patients with AIDS, and nausea and vomiting associated with cancer chemotherapy in patients who have failed to respond adequately to conventional antiemetic treatments.

THC is recognized as the major psychoactive euphoriant responsible for the characteristic intoxication “high” that follows the smoking or ingestion of Cannabis. In fact, high THC doses can produce hallucinogenic effects. In addition to THC, several less potent metabolites and related compounds are found in the Cannabis plant, including the also psychoactive A8-THC and cannabinol (CBN). Another major compound is cannabidiol (CBD), which has antagonistic effects to THC and is a sedative compound.

Several medicinal and recreational cannabis-containing products are available; however, they mainly suffer from the lack of a standardized dosage and difficulty in maintaining consistency in dosing. Specifically, cannabis compounds can be administered by inhalation of smoke or vapors, ingestion of capsules, edibles, or oil extracts, topical application of creams or ointments, and use of nasal sprays. Each delivery method has its own drawbacks. For example, smoking and vaporizing are considered unhealthy, inconvenient, and lack proper dosage control. Similarly, for forms such as oils, creams, or sprays, the dosage is difficult to control.

Although cannabis has a high margin of safety, it can produce negative side effects. At higher doses in humans, effects can include altered body image, auditory and/or visual illusions, and pseudo-hallucinations. In some cases, in humans, cannabis can lead to dissociative states such as depersonalization and derealization. Occasionally, heavy use, or use by inexperienced human consumers, particularly in an unfamiliar environment, can result in very negative experiences. It is therefore crucial to control the consumed dosage.

The variability in the amount of THC present in any given cannabis product, whether it is a smokable product, an oil, or an edible, is a significant drawback for medical patients, as well as recreational cannabis users. Because of this variability, it is often difficult for new cannabis users to correctly gauge the appropriate amount of cannabis to consume, and likewise it is often difficult for medical patients to accurately dose themselves with the proper amount of THC, CBD or other cannabinoids to address their symptoms. As such, there is a need for a product that enables a consumer to use an accurate, standardized dose of cannabis compounds.

Although oral delivery of solid formulations can offer control of dosing, these may not be suitable for patients who have difficulty swallowing an oral medication in the solid form (e.g., tablets and hard gelatin capsules). These patients mainly include, elderly (who have difficulties taking conventional oral dosage forms because of hand tremors and dysphagia), pediatric patients (who are often fearful of taking solid oral dosage forms) and others which include the mentally ill, developmentally disabled, patients who are uncooperative, on reduced liquid-intake plans or nauseated, and travelers who may not have access to water. In this regard, liquids, syrups, or suspensions are the alternative; however, such formulations lack standardized consistent dosing.

### SUMMARY OF THE DISCLOSURE

Disclosed is an oral film dosage form for lipophilic actives having low solubility in water.

The film layer can be configured for oral transmucosal and oral delivery of the active agent(s).

The film formulation disclosed herein is suitable for lipophilic cannabinoids.

According to some aspect of the disclosure, the oral film dosage form comprises either synthetic cannabinoid such as THC or cannabinoid such as THC extracted from the cannabis plant in combination or not with other cannabinoid like cannabidiol.

According to some aspect of the disclosure, cannabis oil is used to introduce cannabinoids to the film formulation.

Also disclosed is a method to produce oral films containing stable oil-in-water emulsions, in which lipophilic actives are solubilized in the oil phase of an emulsion.

The methods disclosed herein produces OFs containing up to 40% (wt/wt) of oil phase.

In other embodiments, the OFs contain up to 40% (wt/wt) of the oil phase combined with the lipophilic active(s). This enhances the amount of lipophilic actives included in the film formulation while preserving the film characteristics.

The methods disclosed herein require the use of surfactant(s) in amounts no more than 50% of the oil phase, preferably no more than 20% of the oil phase, and more preferably no more than 10% of the oil phase determined by weight of the component.

The OFs disclosed herein preferably contain at least 40% (wt/wt) film-forming polymers.

The formulation disclosed herein allows manufacture of OFs containing up to 20% (wt/wt) of lipophilic active(s).

### BRIEF DESCRIPTION OF THE FIGURES

FIG. 1 illustrates methods for preparing water-based oral films (OFs) containing a lipophilic active(s) diluted/dissolved in a carrier oil. The process involves combining an oil-in-water emulsion with film-forming polymers then casting and drying.

FIG. 2 illustrates measurement of surface wettability/hydrophobicity using contact angle (θ).

FIG. 3 illustrates contact angle measurement of OFs having different surface wettability/hydrophobicity.

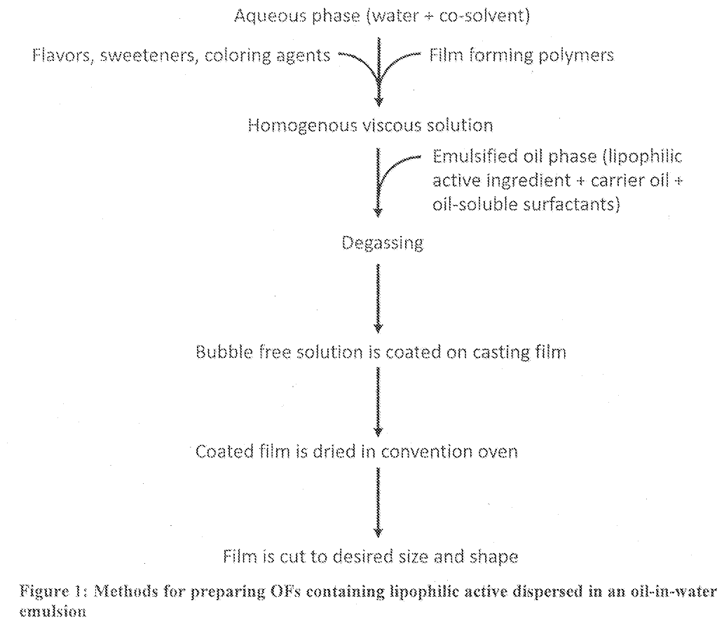

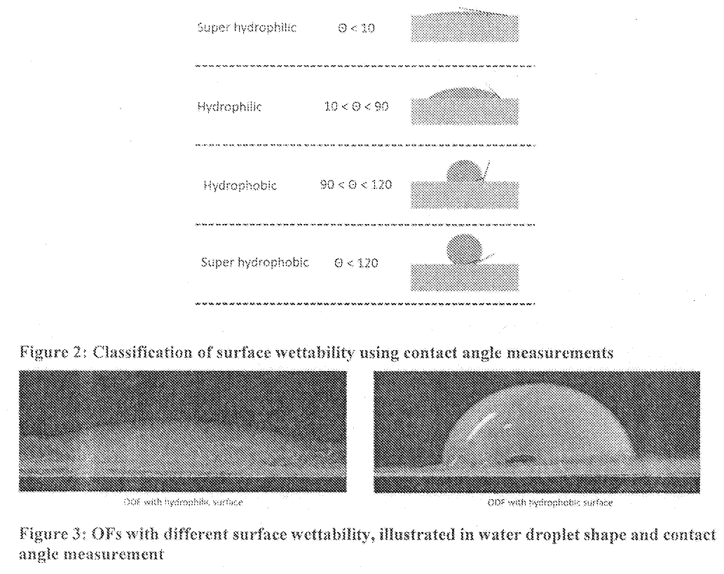

### DETAILED DESCRIPTION OF ILLUSTRATIVE EMBODIMENTS

The terms “oral dissolving film,” “oral dissolvable film”, “oral disintegrating film”, OSF, “oral soluble film”, “ODF”, oral chewable film, “OCF”, “oral thin film”, “OTF,” “oral drug strip’ or “oral strip’ refer to a product used to administer a predetermined amount of active ingredient(s) via oral administration such as oral transmucosal absorption, sublingual delivery or buccal delivery and will be referred to throughout as “oral film” (OF).

The term “OCF” refers to a type of oral film that is orally administered and designed to be chewed by the subject or patient.

The term “film” refers to a type of dosage form that is distinctly different from pills, tablets, caplets, and capsules, and in which the dosage form is a thin strip of material. Such films are typically rapidly disintegrating or rapidly dissolving, but can also exhibit longer disintegration time when required. The films are generally sufficiently flexible to allow bending or even folding without breaking. The films typically have length and width dimensions on the order of 5 to 35 mm, although larger or smaller dimensions are possible and may be desirable in particular circumstances, and a thickness on the order of 5 to 300 μm, although larger or smaller thicknesses are possible and may be desirable in certain circumstances.

The term “active(s)” or “active agent(s)” refers mainly to active pharmaceutical ingredients (APIs), but may also refer generally to any agent(s) that chemically interacts with the subject to which it is administered to cause a biological change, such as, but not limited to, eliminating symptoms of disease or regulating biological functions.

The term lipophilic refers to good oil solubility and/or poor aqueous solubility of a substance. In the present disclosure, for example, the aqueous solubility of a lipophilic active at 37° C. is not more than 10 mg/L, preferably not more than 1 mg/L, more preferably not more than 0.5 mg/L.

The present invention provides a formulation, an OF, suitable for lipophilic APIs.

Examples of lipophilic APIs with low aqueous solubility are: acitretin, albendazole, albuterol, aminoglutethimide, amiodarone, amlodipine, amphetamine, amphotericin B, atorvastatin, atovaquone, azithromycin, baclofen, beclomethasone, benezepril, benzonatate, betamethasone, bicalutanide, budesonide, bupropion, busulfan, butenafine, calcifediol, calcipotriene, calcitriol, camptothecin, candesartan, capsaicin, cannabidiol (CBD), cannabinol (CBN), cannabigerol (CBG), cannabichromene (CBC), cannabichromevarin (CBCV), cannabidivarin (CBDV), cannabigerol monomethyl ether (CBGM), cannabigerovarin (CBGV), cannabielsoin (CBE), cannabicyclol (CBL), cannabivarin (CBV), cannabicitran (CBT), carbamezepine, carotenes, celecoxib, cerivastatin, cetirizine, chlorpheniramine, cholecalciferol, cilostazol, cimetidine, cinnarizine, ciprofloxacin, cisapride, clarithromycin, clemastine, clomiphene, clomipramine, clopidogrel, codeine, coenzyme Q10, cyclobenzaprine, cyclosporin, danazol, dantrolene, dexchlorpheniramine, diclofenac, dicoumarol, digoxin, dehydroepiandrosterone, dihydroergotamine, dihydrotachysterol, dirithromycin, donezepil, efavirenz, eprosartan, ergocalciferol, ergotamine, essential fatty acid sources, etodolac, etoposide, famotidine, fenofibrate, fentanyl, fexofenadine, finasteride, fluconazole, flurbiprofen, fluvastatin, fosphenytoin, frovatriptan, furazolidone, gabapentin, gemfibrozil, glibenclamide, glipizide, glyburide, glimepiride, griseofulvin, halofantrine, ibuprofen, irbesartan, irinotecan, isosorbide dinitrate, isotretinoin, itraconazole, ivermectin, ketoconazole, ketorolac, lamotrigine, lansoprazole, leflunomide, lisinopril, loperamide, loratadine, lovastatin, L-thryroxine, lutein, lycopene, medroxyprogesterone, mifepristone, mefloquine, megestrol acetate, methadone, methoxsalen, metronidazole, miconazole, midazolam, miglitol, minoxidil, mitoxantrone, montelukast, nabumetone, nalbuphine, naratriptan, nelfinavir, nifedipine, nisoldipine, nilutanide, nitrofurantoin, nizatidine, omeprazole, oprevelkin, oestradiol, oxaprozin, paclitaxel, pantoprazole, paracalcitol, paroxetine, pentazocine, pioglitazone, pizofetin, pravastatin, prednisolone, probucol, progesterone, pseudoephedrine, pyridostigmine, rabeprazole, raloxifene, repaglinide, rifabutine, rifapentine, rimexolone, ritanovir, rizatriptan, rofecoxib, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, tetrahydrocannabinol, THC, tetrahydrocannabivarin (THCV), tiagabine, ticlopidine, tirofibran, tizanidine, topiramate, topotecan, toremifene, tramadol, tretinoin, troglitazone, trovafloxacin, ubidecarenone, valsartan, venlafaxine, verteporfin, vigabatrin, Vitamin A, Vitamin D, Vitamin E, Vitamin K, zafirlukast, zileuton, zolmitriptan, zolpidem, and zopiclone. Salts, isomers and derivatives of the above-listed hydrophobic APIs may also be used, as well as mixtures thereof.

The term “cannabinoid” represents a group of C21 terpenophenolic compounds found uniquely in cannabis plants. Cannabinoids include the psychoactive compounds Δ9-tetrahydrocannabinol (THC), Δ8-THC, cannabinol (CBN), 11-hydroxy Δ9-THC, anandamide, and the non-psychoactive compounds cannabidiol (CBD), cannabichromene, and (−) Δ8-THC-11-oic acid. Cannabinoids can be synthetically made or can be extracted from the cannabis plant. The term cannabinoid is used herein to refer to cannabinoid that is either synthetic or extracted from the plant. It is also used to refer to a single cannabinoid or mixture of cannabinoids.

The term “cannabis” is used to refer to plants of the genus Cannabis, including Cannabis sativa and Cannabis indica.

Montelukast as used herein is referring to the protonated form of Montelukast which is lipophilic active with a log P of 7.8.

The term “film former polymers” refers to are water-soluble or water dispersible polymers of common pharmaceutical use that conform to the required properties, including, but not limited to, film instant hydration potential, mucoadhesion and solubility over time. Examples of film forming polymers include cellulose derivatives, polyvinyl alcohol, polyvinyl pyrrolidone, polyethylene oxide, starches, polyacrylates, gums (xanthane gum, arabic gum, guar gum, etc.) and/or mixtures thereof. Film forming polymers may be used in combinations chosen based on the desired characteristics of the delivery form (e.g., rapid disintegration, higher mucoadhesion, longer residence time, etc.). Some of the film forming polymers may also exhibit a surfactant property.

The term “food-grade material” refers to material that is either safe for human consumption or it is authorized by the health authorities or at least one health authority to come into direct contact with food products. Food-grade material herein includes food-grade polymers, surfactants, oils or any other food grade material suitable for oral film manufacturing.

The term “food safe material” means that a food-grade material is also suitable for its intended use and will not create a food-safety hazard.

The lipophilic actives (e.g., lipophilic cannabinoids) used in the water-based OF formulations disclosed herein are diluted/dissolved in an oil vehicle (i.e., a carrier oil) before being introduced into an oil-in-water emulsion. The term “carrier oil” is used to describe a hydrocarbon liquid substance that is typically greasy to the touch, generally formed by natural resources or the breakdown of fats, serves the purpose of diluting a lipophilic active ingredient. The term carrier oil is derived from the purpose of carrying the active ingredient into the formulation. The carrier oil facilitates high loading of lipophilic, poorly water soluble APIs.

Lipophilic active is also understood to refer to actives with a positive log P value. Partition coefficient (P) describes the propensity of a neutral (uncharged) compound to dissolve in an immiscible biphasic system of lipid (i.e., fats, oils, organic solvents) and water. The P value measures how much the compound dissolves in the water portion versus the lipid portion. The log P value is defined as: log P=log [lipophilic active in lipid phase]/[lipophilic active in aqueous phase]. A negative value for log P means the compound has a higher affinity for the aqueous phase (i.e., it is more hydrophilic). A log P of 0 represent that the compound is equally partitioned between the lipid and aqueous phases while a positive value for log P denotes a higher concentration in the lipid phase (i.e., the compound is more lipophilic). A log P of 1 means that there is a 10:1 ratio of the active in the lipid phase to the aqueous phase.

The OF formulations disclosed herein are water based, and include an oil-in-water emulsion. The emulsion is typically made up of oil phase, water phase, and surfactant(s)/emulsifier(s).

The OF formulation disclosed is suitable for oral delivery of lipophilic cannabinoids. Administering cannabis compounds using the disclosed OFs improve administration convenience, mitigates dosage uncertainty, and improves patient acceptability when compared to other known cannabinoid method of administration such as pills, tablets, smoking, vaping, and some available edibles.

According to the disclosure, lipophilic cannabinoids that can be formulated into OFs as disclosed herein include THC, and other cannabinoid derivatives or a mixture thereof, which, in their pure form, are viscous oil of high lipid solubility and low aqueous solubility (i.e. for THC solubility in water 2.8 mg/mL, log P 7.29).

According to certain embodiments, the OF formulations described herein use carrier oil (one type of oil or a mixture of oils) to help bring down the overall viscosity of cannabinoids, such as THC with or without other cannabinoids, thus easing handling requirement during manufacture and promoting the production of water-compatible mixtures and formulations. Cannabinoid lipophilicity, viscous nature, and chemical instability (THC is susceptible to decomposition by oxidation, heat, acid, and light) is generally an impediment to the development of commercially viable and effective formulation for human and animal administration.

CBD is another example of lipophilic cannabinoids that can be formulated into OFs as disclosed herein. Similar to THC, CBD is poorly soluble in water, but is soluble in oil due to its high lipophilicity (solubility in water 0.0126 mg/mL, log P 6.1).

The water-based OF formulations disclosed herein are also suitable for cannabis isolates (e.g., THC oil extract or CBD oil extract or THC/CBD oils) as well as full spectrum cannabis extracts (i.e., combinations of cannabinoids and terpenes). The formulations are also suitable for synthetic cannabinoids and their derivatives.

In accordance with certain aspects of this disclosure, the carrier oil can be, but not limited to, almond oil, apricot kernel oil, avocado oil, borage seed oil, camellia seed oil, caprylic/capric triglycerides, castor oil (or hydrogenated castor oil), coconut oil, cranberry seed oil, cocoa butter (e.g., deodorized cocoa butter oil), corn oil, grapeseed oil, hazelnut oil, hemp seed oil, macadamia nut oil, olive oil, peanut oil, pecan oil, perilla oil, pine nut oil, pistachio oil, poppy seed oil, pumpkin seed oil, rice bran oil, safflower oil, sesame oil, shea butter, soybean oil, sunflower oil, walnut oil, or watermelon seed oil.

According to the disclosure, the carrier oil can be mixtures of mono-, di-and tri-fatty acid esters of glycerol, and mono- and di-fatty acid esters of polyethylene glycol, known as polyoxyethylated fatty acid glycerides. Polyoxyethylated fatty acid glycerides can be prepared by esterification of glycerol and polyethylene glycol with fatty acids. The polyethylene glycol used can have an average of 6 ethylene oxide units (e.g., PEG-6, also referred to as MACROGOL-6). The fatty acids that can be used include, for example, oleic acid, lauric acid and lionleic acid. A specific example of a suitable mixture of polyoxyethylated fatty acid glycerides is oleoyl polyoxy-6 glycerides (also known as oleoyl macrogol-6 glycerides and PEG-6 glyceryl oleates), which is a mixture of mono-, di-and tri-oleic acid esters of glycerol and mono- and di-oleic acid esters of polyethylene glycol (PEG-6). Oleoyl polyoxy-6 glycerides also referred to as Apricot kernel oil PEG-6 esters are commercially available as Labrafil® M 1944 CS (Gattefosse Corporation, Paramus, N.J.). Another example of a suitable mixture of polyoxyethylated fatty acid glycerides that can be used as a carrier oil in the disclosed oral film dosage forms is linoleoyl polyoxyl-6 glycerides (also known as lineoleoyl macrogol-6 glycerides and PEG-6 glyceryl linoleates), which is a mixture of mono-, di- and tri-linoleic acid esters of glycerol and mono- and di-linoleic acid esters of polyethylene glycol (PEG-6). Linoleoyl polyoxyl-6 glycerides are commercially available as Labrafil® M2125 CS (Gattefosse Corporation, Paramus, N.J.). Another example of a mixture of polyoxyethylated fatty acid glycerides that may be useful in the disclosed oral film dosage forms is lauroyl polyoxyl-6 glycerides (also known as lauroyl macrogol-6 glycerides and PEG-6 glyceryl laurates), which is a mixture of mono-, di- and tri-lauric acid esters of glycerol and mono- and di-lauric acid esters of polyethylene glycol (PEG-6). Lauroyl polyoxyl-6 glycerides are commercially available as Labrafil® M2130 CS (Gattefossé Corporation, Paramus, N.J.). Mixtures of any of the foregoing or other polyoxyethylated fatty acid glycerides may be used in the disclosed oral film dosage forms.

According to embodiments, the amount of carrier oil in formulations is preferable higher than the film content of the lipophilic active(s). According to one embodiment, 50% or more of the film oil content is carrier oil. The carrier oil is dissolves the lipophilic active(s) and promote its incorporation within the film matrix. The methods disclosed herein are designed for manufacturing or producing OFs containing an oil phase.

According to some embodiments, up to 40% (wt/wt) of the OF content is the oil phase, amounting of up to 40% determined by weight of oil component per layer. Dosage strength may be increased by using the multilayer film approach, by having an OF comprising a plurality of layers such as a bilayer film, a trilayer film and other type of multilayer as long as the thickness of the film is not negatively impacting the ease of administration. In other embodiments, the oil phase combined with the lipophilic active(s) makes up to 40% (wt/wt) of the OFs composition. The carrier oil used in the emulsion formulation process is a lipid, which provides a great practicality for loading lipophilic/poorly water soluble actives. The ability to load high oil content in a OF is desirable, especially if it allows incorporation of higher content of lipophilic active agent(s).

The OF products of the present disclosure are capable of accommodating a wide range of amounts of the lipophilic active ingredient. The OFs are capable of providing an accurate dosage amount (determined by the size of the film and concentration of the active in the original oil in water emulsion) regardless of whether the required dosage is high or extremely low. Therefore, depending on the type of active or pharmaceutical composition that is incorporated into the film, the active amount may be as high as about 100 mg, desirably up to about 50 mg, more desirably up to 40 mg or as low as the microgram range, or any amount therebetween.

The OF products and methods of the present invention are well suited for high potency, low dosage drugs. This is accomplished through the high degree of uniformity of the films and stability of the lipophilic active through the oil in water emulsion.

The methods disclosed herein comprise the use of surfactant(s) in amounts no more than 50% of the oil phase (wt/wt), preferably no more than 20% of the oil phase, and more preferably no more than 10% of the oil phase.

The OFs disclosed herein preferably also contain at least 40% (wt/wt) film-forming polymers.

Incorporating lipophilic active(s)/carrier oil in water-based OFs is achieved by dispersing the excipients in water. This dispersion is promoted by the use of at least 2 percent, preferably at least 5% and more preferably more than 10%, and most preferably more than 15% of surfactants. The term “surfactant” refers to surface-active agents that possess both polar (hydrophilic) and non-polar (hydrophobic, lipophilic) characteristics in the same molecule. Surfactants are emulsifying agents capable of adsorbing to the oil-water interface and forming a protective coating around droplet aggregations in the oil/water mixture. For lipophilic active ingredients that are poorly soluble in water, the use of surfactants reduces the interfacial tension between the aqueous medium and the lipophilic active(s) thereby increasing their solubility and water compatibility.

Examples of surfactants/emulsifying agents with long chain aliphatic amines or amine salts, partial esters of polyhydrie alcohols, alcohol sulphates, hydrocarbon sulphonic acids, lecithin, or various commercial emulsifiers suitable for use in oral products include, but are not limited to, tween and span, phospholipids (egg, soy, or dairy lecithin), amphiphilic proteins (e.g., whey protein isolate, caseinate), and amphiphilic polysaccharides (eg, gum Arabic, modified starch).

According to some embodiments, other surfactants can also be used, in vivo, to enhance penetration and/or wettability of the film to promote adhesion, those surfactant include polysorbates (Tween™), sodium dodecyl sulfate (sodium lauryl sulfate), lauryl dimethyl amine oxide, cetyltrimethylammonium bromide (CTAB), polyethoxylated alcohols, polyoxyethylene sorbitan octoxynol (Triton X100™), N,N-dimethyldodecylamine-N-oxide, hexadecyltrimethylammonium bromide (HTAB), polyoxyl 10 lauryl ether, Brij 721™, bile salts (sodium deoxycholate, sodium cholate) polyoxyl castor oil (Cremophor™, nonylphenol ethoxylate (Tergitol™), cyclodextrins, lecithin, methylbenzethonium chloride (Hyamine™).

The surfactants used in OF formulations disclosed herein can comprise blends of oil-soluble and water-soluble surfactants.

OFs can offer a standardized dosage form as well as easier and more convenient and discreet administration, transportation, handling, and storage. OFs administration help in mitigating risks of choking and while also alleviating some concern with product friability. OFs are taken with or without water. OFs can be taken without water due to their ability to dissolve and/or disintegrate relatively quickly to releasing the active(s) in the mouth or allow permeation of the active through the mucosa or in the Gastro intestinal tract (GIT). OFs by design thereby promote patient and subject safety and acceptability. OFs offer an attractive route for delivering cannabinoids whether derived or not from cannabis. However, currently available films or wafers containing cannabis extracts or cannabis compounds lack consistency, product dosage homogeneity, and good physical characteristics (e.g., non-stickiness, non-tackiness, uniform appearance, and ease of peelability from substrate). An additional challenge with the integration of cannabinoids in OFs arise from their typically viscous oils characteristic when in concentrated forms at room temperature and normal pressure. This presents some additional difficulties for their incorporation into water-based formulations, such as OFs. The resulting films are often very sticky, and not easily handled and/or packaged, thus making the product not suitable for large scale production and/or not available at a commercially acceptable price. Examples of such OF formulations are given in Tables 1 and 2. Formulations 1 and 3 are examples of a Montelukast- and THC-containing OF, formulated without the inclusion of a carrier oil. This OF is characterized to be sticky. On the other hand, Formulations 2 and 4 are examples of OFs formulated with Montelukast and THC diluted in a carrier oil. In Formulations 2 and 4, the resulting OF is not sticky. The present disclosure incorporates Montelukast and lipophilic cannabinoids such as THC which is a viscous oil, with high lipid solubility and low aqueous solubility, in water-based OF formulations by diluting the cannabinoid in a carrier oil and dispersing in water using surfactants. The carrier oil reduces the overall viscosity of cannabinoid mixture or extract, making it easier to handle and incorporate in water-based OF formulations. The presence of carrier oil in the formulation resulted in reducing film stickiness or adhesiveness, making the film suitable for large scale production and commercial exploitation. An example of lower adhesiveness (adhesiveness is understood as the ability of the film to adhere to surfaces) OF formulations is illustrated in Formulations 2 and 4, Table 1.

TABLE 1
An OF formulation containing Montelukast.

| | Formulation | | Formulation | |
|---|---|---|---|---|
| | 1-sticky OFs | | 2-non-sticky OFs | |
| | (% wt/wt) | (% wt/wt) | (% wt/wt) | (% wt/wt) |
| Excipient | Wet Blend | Dry Film | Wet Blend | Dry Film |
| Water | 82.03 | — | 79.82 | — |
| Pullulan | 9.60 | 53.41 | 9.53 | 47.27 |
| Xanthan gum | 0.08 | 0.45 | 0.08 | 0.45 |
| Locust bean | 0.08 | 0.45 | 0.08 | 0.45 |
| gum | | | | |
| Carrageenan | 0.80 | 4.50 | 0.80 | 4.50 |
| Sucralose | 1.09 | 6.01 | 1.09 | 5.34 |
| glycerin | 1.92 | 10.68 | 1.91 | 9.36 |
| Sorbitol | 0.64 | 3.56 | 0.64 | 3.09 |
| Tween 80 | 1.23 | 6.85 | 1.22 | 5.95 |
| Span 80 | 0.72 | 4.00 | 0.71 | 3.57 |
| Montelukast | 1.81 | 10.09 | 2.06 | 10.01 |
| MCT Oil | 0 | 0 | 2.06 | 10.01 |
| Total Mass | 100 | 100 | 100 | 100 |

TABLE 2
An OF formulation containing THC
(extracted and purified from cannabis plant).

| | Formulation | | Formulation | |
|---|---|---|---|---|
| | 3-sticky OFs | | 4-non-sticky OFs | |
| | (% wt/wt) | (% wt/wt) | (% wt/wt) | (% wt/wt) |
| Excipient | Wet Blend | Dry Film | Wet Blend | Dry Film |
| Water | 82.03 | — | 79.82 | — |
| Pullulan | 9.60 | 53.41 | 9.53 | 47.27 |
| Xanthan gum | 0.08 | 0.45 | 0.08 | 0.45 |
| Locust bean | 0.08 | 0.45 | 0.08 | 0.45 |
| gum | | | | |
| Carrageenan | 0.80 | 4.50 | 0.80 | 4.50 |
| Sucralose | 1.09 | 6.01 | 1.09 | 5.34 |
| glycerin | 1.92 | 10.68 | 1.91 | 9.36 |
| Sorbitol | 0.64 | 3.56 | 0.64 | 3.09 |
| Tween 80 | 1.23 | 6.85 | 1.22 | 5.95 |
| Span 80 | 0.72 | 4.00 | 0.71 | 3.57 |
| THC | 1.81 | 10.09 | 2.06 | 10.01 |
| MCT Oil | 0 | 0 | 2.06 | 10.01 |
| Total Mass | 100 | 100 | 100 | 100 |

According to embodiments, viscous THC oil with high lipid solubility and low aqueous solubility, is incorporated in water-based formulations such as OFs by first diluting/dissolving the THC in a carrier oil, and then using surfactant(s)/emulsifying agent(s) to disperse the oil in water.

Dispersing the oil in water using surfactants/emulsifying agents results in an oil-in-water emulsion. An emulsion is generally defined as two immiscible liquids with one of the liquids being dispersed as spherical droplets within the other. When the two liquids are oil and water and when the oil phase is dispersed in the water phase, the system is called an oil-in-water emulsion.

Preparation of emulsions typically requires oil, water, surfactant(s)/emulsifying agent(s), and energy input. The energy input is commonly provided by mechanical forces applied to the system in the form of shear, turbulence, or cavitation, most commonly using high-pressure homogenization or sonication devices. These are high-energy methods that generate intense disruptive forces that mechanically breakup the oil phase into tiny droplets that are dispersed within the aqueous medium. There are a number of drawbacks in using high-energy methods (e.g., high-pressure homogenization or sonication devices) to produce emulsions, such as high equipment and operating costs and high power requirement. In the case of emulsions containing cannabinoids, an additional drawback to the use of high-energy methods is that they may jeopardize the stability of cannabinoids such as THC. Therefore, to avoid the equipment operating costs and to minimize THC degradation, low-energy methods are preferred for generating cannabinoids emulsions. According to some embodiments, the emulsions are spontaneously formed without the application of high-energy mechanical forces. This is achieved with specific surfactant geometry and concentration, mixing conditions, addition rate, stirring speed and temperature.

For emulsion-based OFs disclosed herein, a stable emulsion (during blending, casting, and drying) is necessary for yielding uniform OFs, in which the oil droplets remain emulsified and stabilized within the dry polymer film matrix. Emulsions can become unstable due to several physicochemical mechanisms such as flocculation, flotation, sedimentation, coalescence, Ostwald ripening and phase inversion. These destabilizing mechanisms are related. For example, there is an increase in particle size due to aggregation by flocculation, coalescence or Ostwald ripening. This results in an increase in droplet instability and thus leads to gravitational separation (flotation/sedimentation). Additionally, these processes may happen simultaneously or consecutively.

For OFs disclosed herein, the composition and total amount of oil phase (i.e. carrier oil, lipophilic active(s), and oil-soluble surfactants/emulsifiers) will generally impact the ability to initially create the emulsion within which the oil droplets are well dispersed in the aqueous phase, while typically also influencing the subsequent stabilization of the emulsion against destabilizing mechanisms (e.g., Ostwald ripening).

Specifically, larger differences in the viscosity between the oil and the aqueous phases will hinder the emulsion formation and promote phase separation. The formulations disclosed herein thus preferably include viscosity modifying agents to increase the viscosity of the aqueous phase and improve the emulsion stability by diminishing the rise of the oil droplets to the surface. Table 3 contain examples of increasing the viscosity of an aqueous solution using glycerol/glycerine. Viscosity modifier are added to the aqueous phase to increase the viscosity in and attempt to mitigate the difference in viscosity between the oil phase and the aqueous phases and thus promote emulsion stability.

Viscosity-modifiers include, but are not limited to, glycerol/glycerin, caprylic/capric triglyceride, propylene glycol dicaprate/dicaprylate, cetearyl alcohol, stearyl alcohol, behenyl alcohol, cetyl alcohol, hydrogenated castor oil, and hydrogenated Shea butter.

TABLE 3
Viscosity of aqueous glycerol solutions. The solutions were
prepared by mixing calculated weights of glycerol and of MilliQ
water. Viscosity was measured using a DV1 Viscometer (CAN-AM
instruments LTD, model DV1MRVTB0, serial #8697375) equipped
with a SC4-21 spindle rotating at the specified values. Measurements
were taken at room temperature (23.5° C.*Theoretical value

| % Glycerol | Viscosity | Spindle | Torque |
|---|---|---|---|
| (wt/wt) | (cP) | Rotation (rpm) | (%) |
| 0%* | 1.0 | N/A | N/A |
| 5% | 1.5 | 100 | 0.4 |
| 10% | 3.0 | 50 | 0.4 |
| 50% | 11.0 | 10 | 0.2 |
| 70% | 20.0 | 10 | 0.3 |
| 100% | 905.0 | 5 | 18.1 |

In certain embodiments, viscosity-modifying agents are added in amounts no more than 10% (wt/wt) of the wet blend formulation, preferably no more than 5% (wt/wt) and more preferably no more than 2.5% (wt/wt).

The effect of viscosity difference (between oil dispersed phase and aqueous continuous phase) on emulsion stability was investigated by preparing oil-in-water emulsion samples of varying the amount of glycerol added (between 0% and 5% [wt/wt] of blend). The destabilization characteristics of prepared emulsions were followed by visually monitoring changes in droplet sizes/distribution and occurrence of droplet flocculation, coalescence, flotation or sedimentation, using light microscopy. As seen in Table 4, the addition of glycerol improves the quality of prepared oil-in-water emulsions.

TABLE 4
Variation of emulsion stability with addition of a viscosity
modifying agent to the aqueous phase. The emulsion consisted
of melted cocoa butter emulsified with lecithin and homogenized
in aqueous glycerol solutions at 5000 rpm for 3 minutes. The
resulting oil in water emulsions were examined by light
microscopy within 1 hour of their preparation.

| Viscosity | | | | | |
|---|---|---|---|---|---|
| Modifying | Emulsion Destabilization Characteristic | | | | |
| Agent % | Droplet | | | | Sedi- |
| (wt/wt) | Size | Flocculation | Coalescence | Flotation | mentation |
| 0% | Mixed | Yes | Yes | Yes | No |
| Glycerol | sizes | | | | |
| 2.5% | Mixed | No | No | No | No |
| Glycerol | sizes | | | | |
| 4.0% | Mixed | Yes | No | No | No |
| Glycerol | sizes | | | | |

The viscosity of lipophilic actives and cannabinoids, such as THC and cannabis oils are generally much different (higher) than that of water. In addition to using viscosity-modifying agents to increase the overall viscosity of the aqueous phase, the disclosed formulations use the carrier oil/lipophilic surfactant(s) to reduce the overall viscosity of the cannabis oil phase, promoting the production of improved stability, making it easier to produce stable THC and cannabis oil-in-water emulsions.

Additionally, in an oil-in-water emulsion, the higher the oil phase viscosity, the larger the minimum achievable droplet size and, therefore, the lower the kinetic stability of the emulsion. It is therefore important to identify an appropriate oil phase (carrier oil and lipophilic emulsifiers) to prepare stable emulsions, especially when applying low-energy methods. The effect of oil phase viscosity on emulsion stability was investigated by preparing oil-in-water emulsion samples of varying viscosity values. The emulsions were prepared using low-energy methods (mixing at 1000 rpm for 10 minutes). The destabilization characteristics of prepared emulsions were followed by monitoring changes in droplet sizes/distribution and occurrence of droplet flocculation, coalescence, flotation or sedimentation, using light microscopy. As seen in Table 5, the higher the oil phase viscosity, the larger the droplet size, and therefore the more unstable the resulting emulsion (seen as floating of oil droplets to the surface, cohesion between oil droplets, and finally to creaming and separation).

TABLE 5
Variation of emulsion stability with oil (dispersed) phase viscosity.
The emulsion consisted of MCT oil emulsified with a mixture of
Tween 80 and Span 80, and mixed in aqueous solution at 1000 rpm
for 10 minutes. The resulting oil-in-water emulsions were examined
by light microscopy within 1 hour of their preparation.

| Viscosity | | | | | |
|---|---|---|---|---|---|
| of oil | O/W Emulsion Destabilization Characteristic | | | | |
| (dispersed) | Droplet | | | | Sedi- |
| phase | Size | Flocculation | Coalescence | Flotation | mentation |
| 30.5 | Very | No | No | Yes | No |
| | small | | | | |
| 35.0 | Very | No | No | No | No |
| | small | | | | |
| 50 | Small | No | No | No | No |
| 60 | Mixed | No | Yes | Yes | No |
| | sizes | | | | |

A lower viscosity for the oil (dispersed) phase can also be achieved by increasing the temperature. In some embodiments initial carrier oil was heated to 70-90° C. in other embodiments, the emulsified oil/surfactant(s) mixture was heated to 70-90° C.

Only certain types and combinations of surfactants/emulsifying agents are suitable for forming emulsions, spontaneously, without the application of high-energy methods. The choice of suitable surfactant(s) starts with determining the hydrophobic lipophilic balance (HLB) value that matches that of the carrier oil in the formulation. The HLB value of a surfactant (or an oil) is a measure of the degree to which it is hydrophilic or lipophilic, determined by calculating values for the different regions of the molecule. In the formulations disclosed herein, the HLB values are chosen so that the surfactants are hydrophilic but able to be soluble in the oil phase. A combination of small-molecule surfactants (such as polysorbate and sorbitan) and phospholipids (such as phosphatidylserine, phosphatidylethanolamine, phosphatidylcholine, and sphingomyelins, egg/soy/dairy lecithin) are found to be the most effective for emulsifying lipophilic active(s) in formulations disclosed herein, using low-energy methods.

In some embodiments, one surfactant (with the same HLB value as the carrier oil) is used. In other embodiments a combination of surfactants (with low and high HLB values) are used.

The surfactant(s)/emulsifying agent(s) concentration is an important factor in the formation and stability of emulsions formed by low-energy methods. When applying low-energy methods (e.g., stir speed 200 to 800 rpm), a relatively high amount of surfactant (e.g., a 1:1 oil:surfactant ratio) is required. This can lead to cost, taste, and safety concerns. Similarly, an increased oil content in the emulsions requires higher amount of surfactant to stabilize an oil-in-water emulsion prepared by low-energy methods. With high content of lipophilic active(s), carrier oil, and surfactant(s) in the oil phase of the emulsion—and in the resulting OFs—it is critical to determine the optimal combination of oil phase components that can produce a stable oil-in-water emulsion while preserving the resulting OF physical/mechanical properties. In the formulations disclosed herein, the amount of surfactant/emulsifying agent required to spontaneously produce emulsions is reduced by using co-solvents (eg, glycerol, propylene glycol, and ethanol). Co-solvents can alter the bulk properties of aqueous solutions (eg, viscosity, see Table 2). The methods disclosed herein allow the use of surfactant(s) in amounts no more than 50% of the oil phase, preferably no more than 20% of the oil phase, and more preferably no more than 10% of the oil phase.

The emulsions disclosed herein are produced based on the spontaneous formation of small oil droplets in surfactant-oil-water system under specific environmental conditions (i.e., composition, temperature, stirring). The oil droplets are then trapped in a polymer matrix in the form of an OF.

According to some embodiments, spontaneous formation of emulsions is attractive because it does not require the use of any specialized homogenization equipment which makes the process more economically efficient and less time consuming when compared with emulsions requiring the use of homogenizer. However, a number of important factors related to the preparation conditions (mixing conditions, addition rate, stirring speed and temperature) must be taken into account to produce stable emulsions, spontaneously. The presently disclosed emulsions are preferably prepared by mixing the aqueous phase with the oil phase. It is preferred to ensure homogeneity of the oil phase prior to mixing the oil phase with the aqueous phase. In addition, the oil phase is expected to be adequately mixed prior to the combination of the oil and aqueous phases. According to embodiments, the lipophilic actives are mixed with the carrier oil and the surfactant(s). The surfactant preferably, display at least slightly hydrophilic chemical characteristics. The components (lipophilic active(s), carrier oil and surfactant) of this oil phase are stirred together to mitigate potential uniformity issues. The oil phase is then titrated into the aqueous phase at a controlled rate until formation of small oil droplets is achieved. Constant mixing or stirring should be maintained for a period suitable to promote homogeneity of the oil in water emulsion.

The emulsified oil phase can be titrated into the aqueous phase at an addition rate of about 15 g per minute, more preferably 10 g per minute. During this addition/titration time, the mixture is continuously stirred at 500 rpm for 60 minutes, preferably 30 minutes, and more preferably 15 minutes.

According to other embodiment, the mixture is continuously stirred at speed of 300 to 700 rpm, preferably 400-600 rpm, more preferably 450-550 rpm with respective time of from 90 to 15 minutes, 80 to 20 minutes, 60 to 40 minutes.

The aqueous phase, into which the oil phase is titrated, contains water and hydrophilic surfactant(s)/emulsifying agent(s). According to some embodiments, it is desirable to add surfactant to the oil phase to aim at bridging the difference in viscosity between the oil and aqueous phases.

According to some embodiments, the aqueous phase may contain co-solvent(s) such as glycerol, propylene glycol, and ethanol.

Additionally, the aqueous phase may contain tonicity agent(s) such as sodium chloride, potassium chloride, mannitol and dextrose.

Additionally, the aqueous phase may contain viscosity modifying agent(s), such as glycerol/glycerin, caprylic/capric triglyceride, propylene glycol dicaprate/dicaprylate, cetearyl alcohol, stearyl alcohol, behenyl alcohol, cetyl alcohol, hydrogenated castor oil, and hydrogenated Shea butter.

Co-solvent(s), tonicity agent(s) are used to promote formation of emulsions with small oil droplets, by modifying the dispersed oil phase and the continuous aqueous phase to have similar viscosities thereby facilitating the rapid movement of surfactant, oil, and water molecules (see Tables 2, 3, and 4). Small size of emulsified oil droplets is desirable because it helps stabilize the oil droplets within the polymer matrix during the drying process, producing homogenous OFs in which the oil droplets are evenly distributed within the polymer matrix of the dry OF

The OFs disclosed herein contain lipophilic active(s), more specifically lipophilic cannabinoids dispersed in a carrier oil and uniformly distributed in the continuously cast film as emulsified oil droplets into a polymer matrix. The term “matrix” or “film matrix” refers to the polymer component or mixture of polymers, which creates the film forming matrix supporting the API within the oral film dosage form.

The OFs can contain, in addition to emulsified lipophilic active(s) and film-forming polymer(s), the following inactive ingredients or excipients: co-solvent such as glycerol, viscosity modifiers such as PEG, sweeteners such as sucralose, surfactants such as lecithin, and colorants or opacifiers such as titanium dioxide. The formulation may further include antimicrobial agents such as methylparaben or propylparaben, preservatives such as butylated hydroxyl toluene (BHT) or alpha-tocopherol, antioxidants such as citric acid or ascorbic acid, and metal chelators such as ethylenediaminetetraacetic acid (EDTA). The antimicrobial, preservatives, and antioxidants are used alone or in combination.

According to some embodiments, additional excipients (such as sweeteners, flavors, and taste masking agents) make less than 2.5% by weight preferably less than 1% by weight of the OF composition.

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, grape, lime and grapefruit and fruit essences including apple, pear, peach, grape, strawberry, raspberry, cherry, plum, pineapple, apricot or other fruit flavors.

Useful flavors or flavoring agents include natural and artificial flavors. These flavorings may be chosen from synthetic flavor oils and flavoring aromatics, and/or oils, oleo resins and extracts derived from plants, leaves, flowers, fruits and so forth, and combinations thereof. Non-limiting flavor oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, including lemon, orange, grape, lime and grapefruit, and fruit essences including apple, pear, peach, strawberry, raspberry, cherry, plum, pineapple, apricot and the like. These flavorings can be used individually or in combination. Commonly used flavors include mints such as peppermint, artificial vanilla, cinnamon derivatives, and various fruit flavors, whether employed individually or in combination. Flavorings such as aldehydes and esters including cinnamylacetate, cinnamaldehyde, citral, diethylacetal, dihydrocarvyl acetate, eugenyl formate, p-methylanisole, and the like may also be used. Further examples of aldehyde flavorings include, but are not limited to acetaldehyde (apple); benzaldehyde (cherry, almond); cinnamicaldehyde (cinnamon); citral, i.e., alpha citral (lemon, lime); neral, i.e. beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 12,6-dimethyl-5-heptenal, i.e. melonal (melon); 2 dimethyloctanal (greenfruit); and 2-dodecenal (citrus, mandarin); cherry; grape; mixtures thereof; and the like.

Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like.

The amount of flavoring employed is normally a matter of preference, subject to such factors as flavor type, individual flavor, and strength desired. The amount may be varied in order to obtain the result desired in the final product. Such variations are within the capabilities of those skilled in the art without the need for undue experimentation. In general, amounts of about 0.1 to about 5 wt % are useful with the practice of the present invention.

Suitable sweeteners include both natural and artificial sweeteners. Non-limiting examples of suitable sweeteners include, e.g.: water-soluble sweetening agents such as monosaccharides, disaccharides and polysaccharides such as xylose, ribose, glucose (dextrose), mannose, galactose, fructose (levulose), sucrose (sugar), high fructose corn syrup, maltose, invert sugar (a mixture of fructose and glucose derived from sucrose), partially hydrolyzed starch, corn syrup solids, and dihydrochalcones; water-soluble artificial sweeteners such as the soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin and the like; dipeptide based sweeteners, such as L-aspartic acid derived sweeteners, such as L-aspartyl-L-phenylalanine methyl ester (aspartame), L-alpha-aspartyl-N-(2,2,4,4-tetramethyl-3-thietanyl)-D-alaninamide hydrate, methyl esters of L-aspartyl-L-phenylglycerin and L-aspartyl-L-2,5,dihydrophenylglycine, L-aspartyl-2,5-dihydro-L-phenylalanine, L-aspartyl-L-(1-cyclohexyen)-alanine, and the like; water-soluble sweeteners derived from naturally occurring water-soluble sweeteners, such as a chlorinated derivatives of ordinary sugar (sucrose), known, for example, as sucralose; and protein based sweeteners such as thaurnatoccous danielli (Thaurnatin I and II).

Also color additives can be used in preparing the OF. Such color additives include food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide.

Other examples of coloring agents include known azo dyes, organic or inorganic pigments, or coloring agents of natural origin. Inorganic pigments include, for example the oxides of iron or titanium. The oxides of iron or titanium are preferably added in concentrations ranging from about 0.001 to about 10%, and more preferably in amounts of about 0.5 to about 3%, based on the weight of all the components.

The variety of additives that can be incorporated into the inventive compositions may provide a variety of different functions. Examples of classes of additives include excipients, lubricants, buffering agents, stabilizers, blowing agents, pigments, coloring agents, fillers, bulking agents, sweetening agents, flavoring agents, fragrances, release modifiers, adjuvants, flow accelerators, mold release agents, granulating agents, diluents, binders, buffers, absorbents, glidants, adhesives, anti-adherents, acidulants, softeners, resins, demulcents, solvents, surfactants, emulsifiers; such as glycerol mono oleate, elastomers and mixtures thereof. These additives may be added with the active ingredient(s).

Useful additives include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses, hydroxyalkylcelluloses, and hydroxyalkylalkylcelluloses, such as methylcellulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose (CMC) and their alkali metal salts; also suitable are phthalated gelatin, gelatin succinate, crosslinked gelatin, shellac, water soluble chemical derivatives of starch, cationically modified acrylates and methacrylates possessing, for example, a tertiary or quaternary amino group, such as the diethylaminoethyl group, which may be quaternized if desired; and other similar polymers.

According to some embodiments, OFs formed with an oil-in-water emulsion, the lipophilic active(s) are solubilized in the oil phase of an oil-in-water emulsion. The emulsion is mixed with film-forming polymers then casted and allowed to dry, forming OFs. Maintaining the emulsion stability (without droplet flocculation, coalescence, flotation, or sedimentation) during the blending and drying processes are critical for controlling both the content of lipophilic active(s) in the OF and the physical/mechanical properties of the films. The film matrix therefore must comprise an appropriate selection of excipients (see Formulation 1-21 below), which together can maintain the oil droplet composition as a homogenous dispersion, and prevents their aggregation and coalescence. When oil droplet aggregation is not controlled and prevented, the oil droplets size will grow and they will phase separate and desorb from the film matrix, thereby accumulating at the surface of the OF. As this occurs, the oil carrying the lipophilic active(s) will be lost to the OF packaging interior surfaces. This product when consumed will no longer meet the label claim API loading.

After blending the emulsion with the polymers and other inactive ingredients, OFs are manufactured by coating the blend as a thin sheet on a liner and drying the coated blend in an oven. According to some embodiment. the lab-scale drying temperature is between 20° C. and 90° C., preferably 30 and 85° C.; and more preferably, the drying temperature is 40 and 80° C.

Emulsion destabilization (creaming, aggregation, and/or coalescence) during the drying process can affect the structure of the emulsion-based OFs by resulting in a concentrated oil layer at the OF surface. The presence or accumulation of an oil layer on the surface of OFs can be monitored by measuring the surface hydrophobicity using contact angle measurements (see Table 6). Contact angles measurements describe the surface hydrophobicity (see FIG. 3) and detect the formation of a concentrated oil layer at the OF surface. Contact angle measurements indicate the stability of oil emulsions in emulsion-based OFs.

TABLE 6
Summary of contact angle measurements of OFs containing
various amounts of oil. The technique of contact angle measurement
is a direct measurement of the tangent angle at the three-phase
contact point on a sessile drop profile. A drop (10 μL of deionized
water) is deposited on the OF surface while mounted on a horizontal
stage. An image is recorded using a USB digital microscope,
equipped with a 1000× continuous zoom. The recorded image is then
analyzed using ImageJ free software, using the contact angle plugin.

| | Contact angle |
|---|---|
| Film Content | (degrees) |
| 0% oil (dry film, wt/wt) | 52 |
| 10% oil (dry film, wt/wt) | 49 |
| 20% oil (dry film, wt/wt) | 41 |
| 30% oil (dry film, wt/wt) | 39 |
| Teflon Reference hydrophobic surface | 102 |

Contact angles measurements describe the surface hydrophobicity (see FIG. 3) and detect the formation of a concentrated oil layer at the OF surface. Contact angle measurements indicate the stability of oil emulsions in emulsion-based OFs.

As seen in Table 6, following the fabrication methods disclosed herein results emulsion-based OFs containing emulsified oil droplets stabilized within the polymer matrix. Contact angle of less than 90 degrees were measured for OFs containing various amounts of oil. This indicates that the OF surface is hydrophilic (i.e., the emulsified oil droplets do not accumulate at the OF surface) and that the film matrix sufficiently stabilizes the oil droplets and prevents their aggregation and coalescing.

To further assess the accumulation of an oil layer on the OF surface, weight change was assessed upon drying off the top and bottom OF surfaces using cleaning wipes (Kimwipes® Low-Lint-1-Ply, 4.4×8.4″). Drying was accomplished by tightly wrapping the OF between the cleaning wipes, for at least 2 hours at either 23° C. or 40° C. (i.e., temperature representative of the OF drying conditions applied during the fabrication). This weight change assessment was performed on OFs fabricated using the methods disclosed herein. A reference OF with an oily surface (not fabricated according to excipients/methods disclosed here) was also assessed. As seen in Table 6, OFs weight change upon drying is below 5%, whereas the oily surface reference OF has weight changes up to 15%. This indicates that the OF fabrication methods disclosed herein produce emulsion-based OFs containing emulsified oil droplets stabilized within the casted polymer matrix.

TABLE 7
Summary of Table 7. Assessment of surface oiliness.
The OF top and bottom surfaces were
dried with cleaning wipes, and the OF weight
change was measured. The OF was tightly wrapped with
Kimwipes ® (Low-Lint -1-Ply, 4.4 × 8.4″) for 2 hours.

| | | | |
|---|---|---|---|
| Initial | Final | Weight | Weight |
| weight | Weight | change | Change % |

Room Temperature Study (23° C.)

| | | | | |
|---|---|---|---|---|
| Oil Content | | | | |
| (dry film, wt/wt) | | | | |
| 0% oil | 57 | 56.95 | −0.05 | 0.09 |
| 10% oil | 61.1 | 60.9 | −0.2 | 0.33 |
| 20% oil | 64.5 | 64.3 | −0.2 | 0.31 |
| 30% oil | 70.9 | 70.5 | −0.4 | 0.56 |
| 27% oil reference film | 172.8 | 161.51 | −11.29 | 6.53 |

Higher Temperature Study (40° C.)

| | | | | |
|---|---|---|---|---|
| Formulation | | | | |
| 0% oil | 60.03 | 58.3 | −1.73 | 2.88 |
| 10% oil | 71.16 | 69.14 | −2.02 | 1.41 |
| 20% oil | 64.2 | 62.4 | −1.8 | 2.80 |
| 30% oil | 76.03 | 74.02 | −2.01 | 2.64 |
| 27% oil reference film | 171.36 | 145.45 | −25.91 | 15.12 |

TABLE 8
An OF formulation containing synthetic THC
dispersed in MCT oil.
Formulation 5

| Excipient | % Wet | % Dry |
|---|---|---|
| Water | 76.42 | — |
| Pullulan | 12.42 | 52.67 |
| Xanthan gum | 0.15 | 0.65 |
| glycerin | 1.24 | 5.27 |
| Sorbitol | 1.24 | 5.27 |
| Tween 80 | 2.48 | 10.53 |
| Sucralose | 0.96 | 0.50 |
| Peppermint oil | 0.25 | 1.05 |
| MCT oil | 2.48 | 10.53 |
| Synthetic THC | 2.35 | 9.97 |
| Total Mass | 100 | — |
| Total Dry Mass | — | 100 |

TABLE 9
An OF formulation containing CBD oil extracted and
purified from cannabis plant then dispersed in MCT oil.
Formulation 6

| Excipient | % Wet | % Dry |
|---|---|---|
| Water | 76.78 | — |
| Glycerin | 1.53 | 6.60 |
| Sucralose | 0.92 | 3.94 |
| Ammonium Glycyrrhizate | 0.46 | 1.97 |
| Flavor herb oil | 0.24 | 1.03 |
| Tween 80 | 1.45 | 6.24 |
| Span 80 | 0.87 | 3.74 |
| Microcystalline cellulose | 3.6 | 12.16 |
| Hydroxy propyl cellulose | 9.8 | 45.6 |
| MCT oil | 2.61 | 11.23 |
| CBD oil | 1.74 | 7.49 |
| Total Mass | 100 | — |
| Total Dry Mass | — | 100 |

TABLE 10
An OF formulation containing cannabis oil
dispersed in sesame oil.
Formulation 7

| Excipient | % Wet | % Dry |
|---|---|---|
| Water | 76.39 | — |
| hydrogenated castor oil | 1.10 | 5.40 |
| Pectin | 6.20 | 26.13 |
| Guar gum | 0.80 | 3.33 |
| Sucralose | 0.80 | 3.33 |
| Hydroxylated Lecithin | 2.14 | 8.85 |
| Sesame oil | 10.30 | 43.40 |
| Cannabis oil | 2.27 | 9.56 |
| Total Mass | 100 | — |
| Total Dry Mass | — | 100 |

The OFs described herein are not sticky. They also do not have an oily feeling on the fingers. Most importantly, they are easy to handle and package, and are uniform in content and appearance.

The OFs described herein can be used for convenient delivery of lipophilic pharmaceutical active ingredients or other lipophilic nutritional agents, including essential oils and plant extracts.

According to embodiments, the OF or oral film dosage form comprises more than 20%, more than 25%, more than 30%, more than 35% or more than 40% (wt/wt) of oil.

According to embodiments more than 20%, more than 25%, more than 30%, more than 35% or more than 40% of the total composition of the OF formulation (wt/wt) is a combination of carrier oil and one or a mixture of cannabinoids.

According to embodiments, OF formulations comprise more of the carrier oil than of the lipophilic active or mixture of lipophilic actives.

According to some embodiments, it is disclosed an OCF consisting of: cannabinoids, cannabinoid extracts, cannabinoids derivatives and food grade product.

According to some embodiment, the high oil content OFs have hydrophobic contact angles. High oil content film (up to 40%) have contact angle lower than 90 degrees, preferably lower than 70 degrees, more preferably lower than 50 degrees.

The following are examples of formulations for cannabis-emulsion based OFs.

TABLE 11
The formulation in example 8.
Formulation 8

| Excipient | % Wet | % Dry |
|---|---|---|
| Water | 76.42 | — |
| Pullulan (MW: 200,000) | 12.43 | 52.71 |
| Xanthan gum | 0.15 | 0.64 |
| Glycerin | 1.24 | 5.26 |
| Sorbitol | 1.24 | 5.26 |
| Tween 80 | 2.48 | 10.52 |
| Sucralose | 0.96 | 4.07 |
| Peppermint oil | 0.25 | 1.06 |
| MCT oil | 2.48 | 10.52 |
| Synthetic THC | 2.35 | 9.97 |
| Total | 100.00 | 100.00 |
| Total Dry Mass | 23.58 | |

TABLE 12
The formulation in example 9
Formulation 9

| Excipient | % Wet | % Dry |
|---|---|---|
| Water | 76.78 | — |
| Glycerin | 1.53 | 6.60 |
| Sucralose | 0.92 | 3.94 |
| Ammonium Glycyrrhizate | 0.46 | 1.97 |
| Flavor herb oil | 0.24 | 1.03 |
| Tween 80 | 1.45 | 6.24 |
| Span 80 | 0.87 | 3.74 |
| Microcystalline cellulose | 3.60 | 12.16 |
| Hydroxy propyl cellulose | 9.80 | 45.60 |
| (MW: 200,000) | | |
| MCT oil | 2.61 | 11.23 |
| CBD oil | 1.74 | 7.49 |
| Total | 100.00 | 100.00 |
| Total Dry Mass | 23.22 | |

TABLE 13
Formulation in Example 10
Formulation 10

| Excipient | % Wet | % Dry |
|---|---|---|
| Water | 78.41 | — |
| Hydrogenated Castor Oil | 1.13 | 5.23 |
| Pectin | 6.36 | 29.47 |
| Guar gum | 0.82 | 3.80 |
| Sucralose | 0.82 | 3.80 |
| Hydroxylated Lecithin | 2.20 | 10.17 |
| (Yelkin 1018) | | |
| Cannabis Extract | 10.26 | 47.53 |
| Total | 100.00 | 100.00 |
| Total Dry Mass | 21.59 | |

TABLE 14
Formulation in Example 11
Formulation 11

| Excipient | % Wet | % Dry |
|---|---|---|
| Water | 71.84 | — |
| Ultralec-P | 0.29 | 1.02 |
| Polyethylene glycol 400 | 2.16 | 7.65 |
| Hydroxypropyl cellulose | 10.78 | 38.27 |
| Hydroxypropylmethyl cellulose E5 | 1.44 | 5.10 |
| Hydroxypropylmethyl cellulose E50 | 0.72 | 2.55 |
| Polyethylene oxide N80 | 1.80 | 6.38 |
| Soy Bean Oil | 5.39 | 19.13 |
| Pemulen | 0.22 | 0.77 |
| Cannabis Extract | 5.39 | 19.13 |
| Total | 100.00 | 100.00 |
| Total Dry Mass | 28.16 | |

TABLE 15
Formulation in Example 12
Formulation 12

| Compound | % Wet | % Dry |
|---|---|---|
| Water | 80.97 | — |
| Pectin | 6.60 | 34.65 |
| Microcystalline cellulose (Avicel | 0.84 | 4.42 |
| PH-105 NF I) | | |
| Glycerine | 4.21 | 22.11 |
| Sucralose | 1.26 | 6.62 |
| hydroxylated Lecithin (Yelkin 1018) | 1.14 | 5.97 |
| Cocoa butter | 1.93 | 10.13 |
| Acesulfame Potassium | 0.63 | 3.31 |
| Cannabis Extract | 1.93 | 10.13 |
| Sodium Chloride | 0.51 | 2.66 |
| Total | 100.00 | 100.00 |
| Total Dry Mass | 19.03 | |

TABLE 16
Formulation in Example 13
Formulation 13

| Compound | % Wet | % Dry |
|---|---|---|
| Water | 69.55 | — |
| Glycerine | 1.39 | 4.57 |
| Sucralose | 0.83 | 2.74 |
| Polysorbate 80 | 1.32 | 4.34 |
| Sorbitan Oleate 80 | 0.79 | 2.60 |
| Peppermint Oil | 0.22 | 0.72 |
| Magnasweet | 0.42 | 1.37 |
| MCT Oil | 3.96 | 13.02 |
| Microcystalline Cellulose (Avicel PH-105 NF I) | 11.96 | 39.29 |
| Hydroxypropyl cellulose | 4.45 | 14.62 |
| Ascorbic acid | 0.35 | 1.14 |
| Pullulan (MW: 200,000) | 2.78 | 9.14 |
| Cannabis Extract | 1.96 | 6.44 |
| Total | 100.00 | 100.00 |
| Total Dry Mass (g) | 30.45 | |

TABLE 17
The formulation in example 14.
Formulation 14

| Excipient | % Wet | % Dry |
|---|---|---|
| Water | 76.42 | — |
| Pullulan (MW: 200,000) | 12.43 | 52.71 |
| Xanthan gum | 0.15 | 0.64 |
| Glycerin | 1.24 | 5.26 |
| Sorbitol | 1.24 | 5.26 |
| Tween 80 | 2.48 | 10.52 |
| Sucralose | 0.96 | 4.07 |
| Peppermint oil | 0.25 | 1.06 |
| MCT oil | 2.48 | 10.52 |
| Montelukast | 2.35 | 9.97 |
| Total | 100.00 | 100.00 |
| Total Dry Mass | | 23.58 |

TABLE 18
The formulation in example 15
Formulation 15

| Excipient | % Wet | % Dry |
|---|---|---|
| Water | 76.78 | — |
| Glycerin | 1.53 | 6.60 |
| Sucralose | 0.92 | 3.94 |
| Ammonium Glycyrrhizate | 0.46 | 1.97 |
| Flavor herb oil | 0.24 | 1.03 |
| Tween 80 | 1.45 | 6.24 |
| Span 80 | 0.87 | 3.74 |
| Microcystalline cellulose | 3.60 | 12.16 |
| Hydroxy propyl cellulose (MW: 200,000) | 9.80 | 45.60 |
| MCT oil | 2.61 | 11.23 |
| Montelukast | 1.74 | 7.49 |
| Total | 100.00 | 100.00 |
| Total Dry Mass | 23.22 | |

TABLE 19
Formulation in Example 16
Formulation 16

| Excipient | % Wet | % Dry |
|---|---|---|
| Water | 78.41 | — |
| Hydrogenated Castor Oil | 1.13 | 5.23 |
| Pectin | 6.36 | 29.47 |
| Guar gum | 0.82 | 3.80 |
| Sucralose | 0.82 | 3.80 |
| Hydroxylated Lecithin (Yelkin 1018) | 2.20 | 10.17 |
| Montelukast | 10.26 | 47.53 |
| Total | 100.00 | 100.00 |
| Total Dry Mass | 21.59 | |

TABLE 20
Formulation in Example 17
Formulation 17

| Excipient | % Wet | % Dry |
|---|---|---|
| Water | 71.84 | — |
| Ultralec-P | 0.29 | 1.02 |
| Polyethylene glycol 400 | 2.16 | 7.65 |
| Hydroxypropyl cellulose | 10.78 | 38.27 |
| Hydroxypropylmethyl cellulose E5 | 1.44 | 5.10 |
| Hydroxypropylmethyl cellulose E50 | 0.72 | 2.55 |
| Polyethylene oxide N80 | 1.80 | 6.38 |
| Soy Bean Oil | 5.39 | 19.13 |
| Pemulen | 0.22 | 0.77 |
| Montelukast | 5.39 | 19.13 |
| Total | 100.00 | 100.00 |
| Total Dry Mass | 28.16 | |

TABLE 21
Formulation in Example 18
Formulation 18

| Compound | % Wet | % Dry |
|---|---|---|
| Water | 80.97 | — |
| Pectin | 6.60 | 34.65 |
| Microcystalline cellulose (Avicel PH-105 NF I) | 0.84 | 4.42 |
| Glycerine | 4.21 | 22.11 |
| Sucralose | 1.26 | 6.62 |
| hydroxylated Lecithin (Yelkin 1018) | 1.14 | 5.97 |
| Cocoa butter | 1.93 | 10.13 |
| Acesulfame Potassium | 0.63 | 3.31 |
| Montelukast | 1.93 | 10.13 |
| Sodium Chloride | 0.51 | 2.66 |
| Total | 100.00 | 100.00 |
| Total Dry Mass | 19.03 | |

TABLE 22
Formulation in Example 19
Formulation 19

| Compound | % Wet | % Dry |
|---|---|---|
| Water | 69.55 | — |
| Glycerine | 1.39 | 4.57 |
| Sucralose | 0.83 | 2.74 |
| Polysorbate 80 | 1.32 | 4.34 |
| Sorbitan Oleate 80 | 0.79 | 2.60 |
| Peppermint Oil | 0.22 | 0.72 |
| Magnasweet | 0.42 | 1.37 |
| MCT Oil | 3.96 | 13.02 |
| Microcystalline Cellulose (Avicel PH-105 NF I) | 11.96 | 39.29 |
| Hydroxypropyl cellulose | 4.45 | 14.62 |
| Ascorbic acid | 0.35 | 1.14 |
| Pullulan(MW: 200,000) | 2.78 | 9.14 |
| Montelukast | 1.96 | 6.44 |
| Total | 100.00 | 100.00 |
| Total Dry Mass (g) | 30.45 | |

TABLE 23
The formulation in example 20
Formulation 20

| Excipient | % Wet | % Dry |
|---|---|---|
| Water | 76.42 | — |
| Pullulan (MW: 200,000) | 12.43 | 52.71 |
| Xanthan gum | 0.15 | 0.64 |
| Glycerin | 1.24 | 5.26 |
| Sorbitol | 1.24 | 5.26 |
| Tween 80 | 2.48 | 10.52 |
| Sucralose | 0.96 | 4.07 |
| Peppermint oil | 0.25 | 1.06 |
| MCT oil | 2.48 | 10.52 |
| Tamoxifen | 2.35 | 9.97 |
| Total | 100.00 | 100.00 |
| Total Dry Mass | 23.58 | |

TABLE 24
The formulation in example 21
Formulation 21

| Excipient | % Wet | % Dry |
|---|---|---|
| Water | 76.78 | — |
| Glycerin | 1.53 | 6.60 |
| Sucralose | 0.92 | 3.94 |
| Ammonium Glycyrrhizate | 0.46 | 1.97 |
| Flavor herb oil | 0.24 | 1.03 |
| Tween 80 | 1.45 | 6.24 |
| Span 80 | 0.87 | 3.74 |
| Microcystalline cellulose | 3.60 | 12.16 |
| Hydroxy propyl cellulose (MW: 200,000) | 9.80 | 45.60 |
| MCT oil | 2.61 | 11.23 |
| Tamoxifen | 1.74 | 7.49 |
| Total | 100.00 | 100.00 |
| Total Dry Mass | 23.22 | |

TABLE 25
Formulation in Example 22
Formulation 22

| Excipient | % Wet | % Dry |
|---|---|---|
| Water | 78.41 | — |
| Hydrogenated Castor Oil | 1.13 | 5.23 |
| Pectin | 6.36 | 29.47 |
| Guar gum | 0.82 | 3.80 |
| Sucralose | 0.82 | 3.80 |
| Hydroxylated Lecithin (Yelkin 1018) | 2.20 | 10.17 |
| Tamoxifen | 10.26 | 47.53 |
| Total | 100.00 | 100.00 |
| Total Dry Mass | 21.59 | |

TABLE 26
Formulation in Example 23
Formulation 23

| Excipient | % Wet | % Dry |
|---|---|---|
| Water | 71.84 | — |
| Ultralec-P | 0.29 | 1.02 |
| Polyethylene glycol 400 | 2.16 | 7.65 |
| Hydroxypropyl cellulose | 10.78 | 38.27 |
| Hydroxypropylmethyl cellulose E5 | 1.44 | 5.10 |
| Hydroxypropylmethyl cellulose E50 | 0.72 | 2.55 |
| Polyethylene oxide N80 | 1.80 | 6.38 |
| Soy Bean Oil | 5.39 | 19.13 |
| Pemulen | 0.22 | 0.77 |
| Tamoxifen | 5.39 | 19.13 |
| Total | 100.00 | 100.00 |
| Total Dry Mass | 28.16 | |

TABLE 27
Formulation in Example 24
Formulation 24

| Compound | % Wet | % Dry |
|---|---|---|
| Water | 80.97 | — |
| Pectin | 6.60 | 34.65 |
| Microcystalline cellulose (Avicel PH-105 NF I) | 0.84 | 4.42 |
| Glycerine | 4.21 | 22.11 |
| Sucralose | 1.26 | 6.62 |
| hydroxylated Lecithin (Yelkin 1018) | 1.14 | 5.97 |
| Cocoa butter | 1.93 | 10.13 |
| Acesulfame Potassium | 0.63 | 3.31 |
| Tamoxifen | 1.93 | 10.13 |
| Sodium Chloride | 0.51 | 2.66 |
| Total | 100.00 | 100.00 |
| Total Dry Mass | 19.03 | |

TABLE 28
Formulation in Example 25
Formulation 25

| Compound | % Wet | % Dry |
|---|---|---|
| Water | 69.55 | — |
| Glycerine | 1.39 | 4.57 |
| Sucralose | 0.83 | 2.74 |
| Polysorbate 80 | 1.32 | 4.34 |
| Sorbitan Oleate 80 | 0.79 | 2.60 |
| Peppermint Oil | 0.22 | 0.72 |
| Magnasweet | 0.42 | 1.37 |
| MCT Oil | 3.96 | 13.02 |
| Microcystalline Cellulose (Avicel PH-105 NF I) | 11.96 | 39.29 |
| Hydroxypropyl cellulose | 4.45 | 14.62 |
| Ascorbic acid | 0.35 | 1.14 |
| Pullulan (MW: 200,000) | 2.78 | 9.14 |
| Tamoxifen | 1.96 | 6.44 |
| Total | 100.00 | 100.00 |
| Total Dry Mass (g) | 30.45 | |

According to some embodiment, the OF preferably disintegrate in the mouth or in vitro within 10 min, within 8 minutes, within 6 minutes, within 5 minutes, within 4 minutes, within 3 minutes, within 2 minutes, within 1 minutes.

The OF of the present disclosure must be formed into a sheet prior to drying. After the desired components are combined to form a multi-component matrix, including the polymer, water, carrier oil, surfactant and a lipophilic active, and other components as desired, the combination is formed into a sheet or film, by any method known in the art such as, coating, spreading, casting or drawing the multi-component matrix. A multi-layered film may be achieved by coating, spreading, or casting a combination onto an already formed film layer. Although a variety of different film-forming techniques may be used, it is desirable to select a method that will provide a flexible OF, such as reverse roll coating. The flexibility of the OF allows for the sheets of OF to be rolled and transported for storage or prior to being cut into individual dosage forms. Desirably, the OF will also be self-supporting or in other words able to maintain their integrity and structure in the absence of a separate support. Furthermore, the films of the present invention may use selected materials that are edible or ingestible.

Coating or casting methods are particularly useful for the purpose of forming OF as disclosed herein. Specific examples include reverse roll coating, forward roll coating, gap or knife over roll coating, air knife coating, curtain coating, or combinations thereof, especially when a multi-layered film is desired.

Roll coating, or more specifically reverse roll coating, is particularly desired when forming films in accordance with the present disclosure. This procedure provides excellent control and uniformity of the resulting films, which is desired in the present disclosure. In this procedure, the coating material is measured onto the applicator roller by the precision setting of the gap between the upper metering roller and the application roller below it. The coating is transferred from the application roller to the substrate as it passes around the support roller adjacent to the application roller. Both three roll and four roll processes are common.

According to other embodiments, it may be desirable to have a multilayer OF designed with a first layer comprising a cannabinoid and a second layer having a different cannabinoid. Cannabinoids though similar have differing solubility and lipophilicity. Having a layer comprising a single cannabinoid of a combination of cannabinoids with similar lipophilicity and affinity to a particular oil is desirable for ease in scaling up the manufacturing of large scale OF production.

According to some embodiments, a disclosed OF comprises a first layer having a first cannabinoid and a first carrier oil, and a second layer having a second cannabinoid and a second carrier oil, where the first cannabinoid and the second cannabinoid is different from the first cannabinoid and where the first carrier oil is different than the second carrier oil. This multilayer film further comprises a first surfactant in the first later and a second surfactant in the second layer. In some embodiment the quantity of the first and second surfactants are different. The multilayer approach of manufacturing OF is favored for its ease of manufacture having a targeted formulation for a specific lipophilicity. The optimized liquid formulations are made for 0.01%, 1%, 2%, 3%, 4%, 5%, 6%, 7%, 8%, 9%, 10%, 15%, 20%, 25%, 30% of a specific cannabinoid compound and in concentration of 0.01%, 1%, 2%, 3%, 4%, 5%, 6%, 7%, 8%, 9%, 10%, 15%, 20%, 25%, 30% of a different cannabinoid. This allowed the manufacture of any casting of a multilayer film having a first layer having 10% CBD and subsequently casting a second layer having 10% THC making a combined dosage of a bilayer film with combine dosing of 50/50 CBD/THC. This novel approach would allow for scaling and making a significant amount of different OF for the desired combination and thus meeting the consumer or patient population needs of various combination of therapeutic or recreational effects. The use of this modular approach also reduces cost of production by limiting the necessity of scale up formulation having the combined composition of cannabinoids. For instance, 30 separate blends with each having 0.01%, 1%, 2%, 3%, 4%, 5%, 6%, 7%, 8%, 9%, 10%, 15%, 20%, 25%, 30% of a specific cannabinoid compound and in concentration of 0.01%, 1%, 2%, 3%, 4%, 5%, 6%, 7%, 8%, 9%, 10%, 15%, 20%, 25%, 30% of a different cannabinoid while being able to have up to 225 different oral film dosage forms derived from those 30 blends. In addition, one could make a trilayer film with the third layer having an identical or different cannabinoid allowing for the manufacture of an even greater variation of safe and effective cannabinoid dosing. According to certain embodiments, the OFs have an acidic pH. OFs have a surface pH lower than 7, preferably lower than 5.5, more preferably lower than 4.

According to certain embodiments, the formulation is suitable for chewable/edible OFs. According to some embodiment, OCF formulations contain lipophilic actives and food grade inactive ingredients and comply with all properties of safe-food ingredients according to Food and Drug Administration (FDA) having Generally Recognized As Safe (GRAS) status. Additionally, the edible/chewable OFs or OCF have lower mucoadhesion properties and disintegrate smoothly in the mouth at a moderate rate either with or without actual chewing. OCFs have a smooth texture upon disintegration, are pleasant tasting and leave no bitter or unpleasant taste.

Polymers suitable for formulating OCFs include, but not limited to polypeptides (e.g., collagen and geltain), hydrocolloids (e.g., starch alginate, carrageenan, carboxymethylcellulose, gum arabic, chitosan, pectin, and xanthan gum), lipids (e.g., acetylated monoglycerides, natural wax, and surfactants).

The formulation in Example 5 is suitable for an edible/chewable OF.

The disclosed OF are well suited for many uses. The high degree of desired active uniformity in the OF makes them particularly well suited for incorporating cannabinoids and cannabinoid derivative. Furthermore, the polymers used in construction of the OF may be chosen to allow for a range of disintegration times for the OF. A variation or extension in the time over which a film will disintegrate may achieve control over the rate that the active is released, which may allow for a sustained release delivery system.

The OF are used to orally administer a lipophilic active. This is accomplished by preparing the films as described above and introducing them to the oral cavity of a human or animal, such as a mammal This film may be prepared and adhered to a second or support layer from which it is removed prior to use, i.e. introduction to the oral cavity. An adhesive may be used to attach the OF to the support or backing material which may be any of those known in the art, and is preferably not water soluble. If an adhesive is used, it will desirably be a food-grade material that is ingestible and does not alter the properties of the active.

When designed for animal administration, the OF may desirably be designed to adhere to the oral cavity of the animal including the tongue, thus preventing it from being ejected from the oral cavity and permitting more of the active to be introduced to the oral cavity as the film disintegrates.

Another use for the films of the present invention takes advantage of the tendency of the film to dissolve quickly when introduce to a liquid. An active may be introduced to a liquid by preparing a film in accordance with the present invention, introducing it to a liquid, and allowing it to dissolve. This may be used either to prepare a liquid dosage form of an active, or to flavor a beverage.

The films of the present invention are desirably packaged in sealed, air and moisture resistant packages to protect the active from exposure oxidation, hydrolysis, volatilization and interaction with the environment. Moreover, the films of the present invention dissolve quickly upon contact with saliva or mucosal membrane areas, eliminating the need to wash the dose down with water.

Desirably, a series of such unit doses are packaged together in accordance with the prescribed regimen or treatment, e.g., a 3-90 day supply, depending on the particular therapy. The individual films can be packaged on a backing and peeled off for use.

The above description is considered that of the preferred embodiment(s) only. Modifications of these embodiments will occur to those skilled in the art and to those who make or use the illustrated embodiments. Therefore, it is understood that the embodiment(s) described above are merely exemplary and not intended to limit the scope of this disclosure, which is defined by the following claims as interpreted according to the principles of patent law, including the doctrine of equivalents.

1. An oral film dosage form for human or animal administration comprising:
a film layer comprising;
a safe and effective amount of a lipophilic active;
a carrier oil;
a surfactant; and
a water soluble film forming polymer.

2. The oral film dosage form of claim 1, wherein the combined quantity of carrier oil and lipophilic active is more than about 20% (wt/wt) of the oral film dosage form.

3. The oral film dosage form of claim 1, wherein the combined quantity of carrier oil and lipophilic active is more than about 25% (wt/wt) of the oral film dosage form.

4. The oral film dosage form of claim 1, wherein the combined quantity of carrier oil and lipophilic active is more than about 30% (wt/wt) of the oral film dosage form.

5. The oral film dosage form of claim 1, wherein the combined quantity of carrier oil and lipophilic active is more than about 35% (wt/wt) of the oral film dosage form.

6. The oral film dosage form of claim 1, wherein the combined quantity of carrier oil and lipophilic active is more than about 40% (wt/wt) of the oral film dosage form.

7. The oral film dosage form of claim 1, wherein the film layer further comprises a viscosity modifier.

8. The oral film dosage form of claim 1, wherein the film layer retains at least 95% of the oil and lipophilic active.

9. The oral film dosage form of claim 1, wherein the contact angle of the film is below 90 degrees.

10. The oral film dosage form of claim 1, wherein the contact angle of the film is below 80 degrees.

11. The oral film dosage form of claim 1, wherein the contact angle of the film is below 70 degrees.

12. The oral film dosage form of claim 1, wherein the contact angle of the film is below 60 degrees.

13. The oral film dosage form of claim 1, wherein the lipophilic active includes at least one cannabinoid.

14. The oral film dosage form of claim 1, wherein the carrier oil is a plant extract.

15. The oral film dosage form of claim 1, wherein the carrier oil is a mixture of mono-, di- and tri-fatty acid esters of glycerol.

16. The oral film dosage form of claim 1, wherein the mass of surfactant in the dosage form is less than or equal to 50% of the combined mass of the carrier oil and lipophilic active.

17. The oral film dosage form of claim 1, wherein the mass of surfactant in the dosage form is less than or equal to 10% of the combined mass of the carrier oil and lipophilic active.
